(19) United States
(12) Patent Application Publication
(10) Pub. No.: US 2024/0391618 A1
(43) Pub. Date: Nov. 28, 2024

(54) METHOD OF BUNDLING AND DISPENSING PERSONAL PROTECTIVE EQUIPMENT AND APPARATUS FOR DISPENSING BUNDLED PERSONAL PROTECTIVE EQUIPMENT

(71) Applicant: STAR PPE LLC, Coxs Mills, WV (US)

(72) Inventor: Andrew Jackson, Coxs Mills, WV (US)

(21) Appl. No.: 18/542,403

(22) Filed: Dec. 15, 2023

Related U.S. Application Data
(60) Provisional application No. 63/504,234, filed on May 25, 2023.

(51) Int. Cl. B65B 5/06; A61B 42/40; B65B 31/02

(52) U.S. Cl. CPC B65B 5/067; A61B 42/40; B65B 31/02

(57) ABSTRACT

A method of bundling and dispensing personal protective equipment (PPE) and a dispenser for dispensing the bundled PPE involves the steps of assembling PPE bundles based on PPE component size and type, vacuum sealing the bundles to create planar, flattened packets with a predetermined and uniform shape and size, grouping the packets according to size and component contents, and dispensing the packets in dispensers that keep each packet group separated in one or more dispensers allowing for a single packet to be dispensed.

### CROSS-REFERENCE TO RELATED APPLICATIONS

### Reference to Related Applications

This application is a utility application of and claims priority from U.S. provisional application Ser. No. 63/504,234 filed 25 May 2023, all of which are hereby incorporated by this reference.

### STATEMENT REGARDING FEDERALLY SPONSORED RESEARCH OR DEVELOPMENT

NA

### NAMES OF THE PARTIES TO A JOINT RESEARCH AGREEMENT

NA

### INCORPORATION BY REFERENCE OF MATERIAL SUBMITTED ON A COMPACT DISC OR AS A TEXT FILE VIA THE EFS WEB SYSTEM

NA

### STATEMENT REGARDING PRIOR DISCLOSURES BY THE INVENTOR OR A JOINT INVENTOR

NA

### BACKGROUND OF THE INVENTION

### Field of the Invention

The invention pertains to the field of personal protective equipment (PPE) used by healthcare professionals. Specifically, the invention is a method of bundling disposable PPE into space saving prepackaged packets and dispensing the packets efficiently and hygienically.

### Background Art

Personal protective equipment, or PPE, is used regularly by healthcare professionals in hospitals, medical clinics, and skilled nursing facilities, collectively referred to as healthcare facilities. Often, the PPE used are disposable, single-use isolation gowns, masks, gloves, caps, and shoe coverings or booties. Currently, there is no standardized way to store and dispense the different types of PPE. A survey of different healthcare facilities shows that some have open boxes of PPE sitting on countertops, or positioned inside wall mounted dispensers, where the open box of PPE is slipped into the dispenser. These two storage and distribution methods are most commonly used for disposable gloves and masks. As these are small items, these storage solutions are acceptable, although in the case of gloves, three sizes of gloves must be provided, thus increasing the number of boxes to be stored in each room. Less frequently used PPE, such as isolation gowns, caps, and booties, may also be stored and dispensed in a same way as masks and gloves, but typically, because they are less frequently used, this PPE is less likely to have permanent storage and dispensing methods, and thus tend to be placed “wherever there is room.” There is also no uniformity in box sizes or dispensers, leading further to a cluttered appearance and difficulties with dispensing efficiently.

Wall dispensers are a good solution, but current dispensers are not designed to dispense items like gowns, which are large and easily damaged and thus not suited to be stuffed into a dispenser and pulled out the way a mask or glove can be dispensed. There is no “pop out” gown; the gowns cannot be dispensed the way tissues are dispensed due to size and the risk of damage. If current wall systems are used, it would require a separate dispenser for gloves, gowns, caps, booties, etc. and given that there is limited wall space, realistically there is no room for the plurality of wall dispensers required for all the PPE needed. As PPE necessarily must be kept in a convenient location to allow the health professional access when needed, storing PPE boxes in a central storage location is inconvenient, and storing smaller quantities per room creates extra work for the facility to monitor and stock. Many healthcare facilities are understaffed, so they often just have multiple boxes of PPE in various places where there is room due to this lack of ability to both efficiently and conveniently store and dispense PPE.

The lack of a designated PPE storage and dispensing location means that healthcare professionals must waste valuable time hunting for and then selecting PPE. It is easy for a busy doctor or nurse to forget to take a needed gown or mask, or accidentally take one glove or three gloves instead of two.

The current state of PPE storage and dispensing also creates supply chain issues for the healthcare facility. Each facility will order multiple boxes of small, medium, and large gloves, masks, etc. and then must monitor supplies to ensure timely reordering. There is no easy way to know whether specific types and sizes of PPE supplies are running low, other than doing a manual review. Many different types of PPE must be ordered, as well as different sizes. There is thus no easy or convenient way to monitor supplies. This difficulty tracking PPE inventory results in healthcare facilities overinvesting in inventory, locking up funds that might be used elsewhere, and without any efficient way to track the inventory and thus improve its supply chain.

What is needed is a standardized way of storing and dispensing PPE that is convenient, space saving, hygienic, and efficient. What is also needed is a method of storing and dispensing PPE that is time efficient and provides an easier way for the healthcare facility to control and monitor its PPE inventory.

### Disclosure of Invention

In a first aspect of the invention, a method of bundling and dispensing personal protective equipment for dispensing by a dispenser comprises a bundling step with the steps of selecting personal protective equipment components based on size and type, with type including disposable and reusable components, bundling the selected personal protective equipment components according to the selected personal protective equipment component size and type, and vacuum sealing the bundled personal protective equipment components to form individual personal protective equipment packets with a predetermined uniform size and shape. Size of the personal protective equipment components include small, medium and large sizes, and type of the personal protective equipment are masks, gloves, head coverings, gowns and shoe coverings.

The method further comprises a dispensing step having the steps of grouping the personal protective equipment packets according to packet content, loading each group of personal protective equipment packets into its own dispenser, and dispensing personal protective equipment packets by grasping a personal protective equipment packet from its dispenser.

In a first aspect of the invention, the step of grouping is further comprised of sorting the personal protective equipment packets based on component size and component type.

In a second aspect of the invention, the personal protective equipment packets made using the method are planar with a flattened profile.

In a third aspect of the invention, the personal protective equipment packets have either a tear off strip or a notched edge formed into the packet, whereby the packets are opened by removing the tear off strip or tearing the packet along the notched edge.

In a fourth aspect of the invention, the step of selecting based on component type includes selecting a pair of gloves, a gown, and a mask.

In a fifth aspect of the invention, the step of bundling is further comprised of stacking the personal protective equipment components in a same order prior to the step of vacuum sealing.

In a sixth aspect of the invention, the personal protective equipment packets are dispensed in a dispenser having a dispenser body with a front wall, a back wall, and a side wall defining an interior storage area sized and shaped to receive the personal protective equipment packets, with a dispensing opening adapted as a slot formed into the dispenser body allowing access to the interior storage area. The dispenser body is further comprised of a floor at its lowermost end adjacent the dispensing opening and a lid at its uppermost end, with the floor further comprise of a floor notch and a lip adjacent the dispenser slot. The front wall of the dispenser body is at right angles with the side wall.

In a seventh aspect of the invention, the dispenser is further comprised of an accessory plate having at least one aperture and adapted to be affixed to both the dispenser body and to an accessory attached to the dispenser body.

In a last aspect of the invention, an individual personal protective equipment packet made by the method according to the invention is comprised of personal protective equipment components having a small, medium, large, or single sized component and wherein the personal protective equipment packet includes types of personal protective equipment components that are disposable, reusable, or both disposable and reusable.

### BRIEF DESCRIPTION OF THE DRAWINGS

FIG. 1 is a flow diagram showing a method of bundling and dispensing PPE.

FIG. 2 is a perspective view of a representative PPE packet created by the method in step 1 and used in the method in step 1.

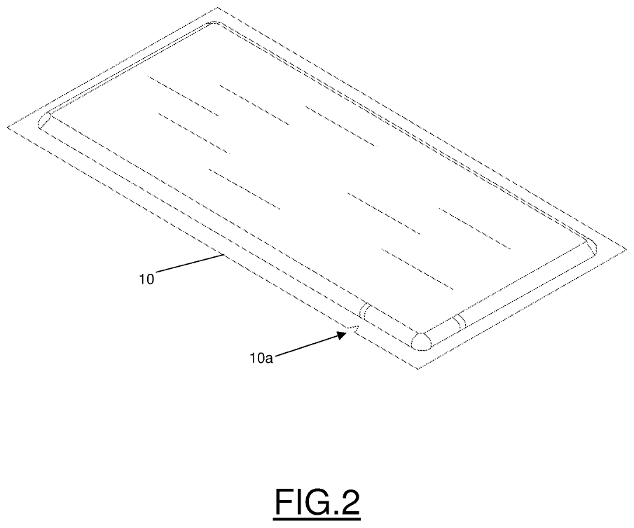

FIG. 3 is a first perspective view of a dispenser having an optional lower shelf and pull out shelf according to the invention, with the dispenser shown empty.

FIG. 4 is a front elevation view of the dispenser in FIG. 3, shown with an optional hand sanitizer bottle and holder, and with a plurality of PPE packets stored inside the dispenser.

FIG. 5 is a cross sectional view of the dispenser in FIG. 4 taken along cut lines A-A.

FIG. 6 is a first perspective view of the dispenser shown in FIG. 4.

FIG. 7 is side elevation view of the dispenser in FIG. 3.

FIG. 8 is a second perspective view of the dispenser in FIG. 3, configured without the optional lower shelf and pull out shelf.

FIG. 9 is a top view of the dispenser in FIG. 3.

FIG. 10 is a perspective exploded view of the optional lower shelf and pull out shelf shown in FIG. 3 attached to the dispenser in FIG. 8.

FIG. 11 is a bottom view of the dispenser in FIG. 8.

### DETAILED DESCRIPTION

An improved method of bundling and dispensing personal protective equipment (PPE) or method 50 and an apparatus for dispensing the bundled PPE or dispenser 20 according to the invention are shown in the accompanying FIGS.

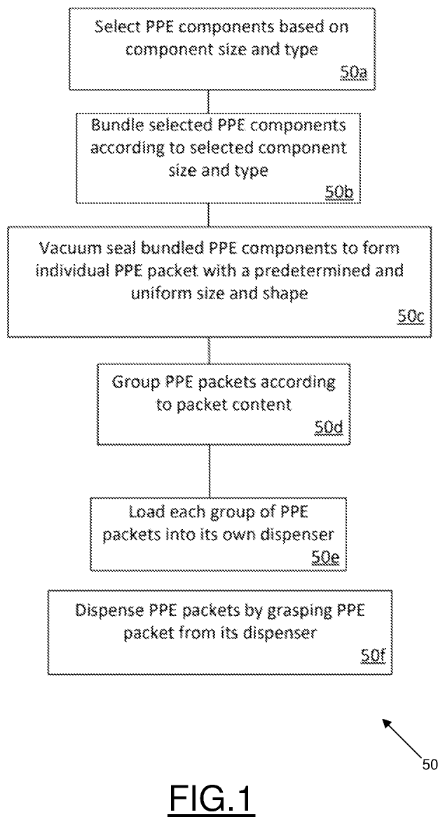

Turning to FIG. 1, the method 50 is comprised of two main steps, a bundling PPE step (substeps 50a to 50c) and then a dispensing step of the bundled PPE (substeps 50d-50f).

Turning to the steps of bundling the PPE, different types of PPE, such as gloves, masks, gowns, caps or head coverings, and shoe coverings or booties, in a first step, the PPE components are selected according to need and size 50a. For instance, in an emergency room setting, a healthcare professional typically needs a pair of gloves, a mask and a gown. Masks and gowns are typically one or a single size, but gloves are commonly sized small, medium, and large. In this selecting step 50a, for the example given, a PPE packet 10 would contain a sized pair of gloves, a mask, and a gown. The selected PPE type can be disposable or reusable, and disposable and reusable PPE can be selected to be bundled into a same PPE packet 10. For instance, a reusable surgical head covering, gown, and disposable mask and gloves can be selected for inclusion into the same PPE packet 10.

In a bundling step 50b, three subsets of glove, mask and gown packets are created: small packets including a pair of small sized gloves, medium packets including a pair of medium sized gloves, and large packets including a pair of large sized gloves. For each subset of packets, the PPE is stacked in a uniform and consistent manner, so for instance, positioning the gloves as a middle layer of a 3 layer PPE packet.

In a next step, the bundles of selected PPE are then vacuum sealed into individual PPE packets 10 with a predetermined uniform size and shape 50c. Ideally, the PPE packets 10 are flat “envelopes” to maximize space and ease of storage and transportation. Disposable PPE is relatively lightweight but tends to be bulky from a volume perspective due to trapped air, and vacuum sealing is an ideal way to shrink storage and transportation requirements while still providing a same quantity of PPE. Reusable PPE is bulkier and must be appropriately cleaned prior to bundling but can still benefit from vacuum sealing to reduce volume. Bundling and prepackaging the PPE into packets 10 simplifies storage, ordering, inventory, and greatly simplifies dispensing, with the additional benefit of reducing accidental damage to PPE commonly caused by current self-dispensing methods from open boxes.

Once the PPE is organized and vacuumed sealed into packets of varying component types and sizes, at the healthcare facility, the PPE packets are grouped according to packet content and size 50d. For instance, all the packets with gowns, masks and small gloves are loaded into a dispenser 20 designated as “small”, all the packets with gowns, masks, and medium gloves are loaded into a second dispenser designated as “medium”, etc. 50e. Ideally, the respective dispensers 20 also provide information as to the PPE packet contents (gloves, masks, gowns), if there are different PPE packet contents. PPE packets with just masks and gloves are not mixed in with PPE packets having masks, gloves, and gowns in a same dispenser 20, according to the method 50 in FIG. 1. The dispenser 20 as shown in the FIGS. is designed for dispensing the PPE packets 10 but the term “dispenser” used in this disclosure includes dispensers not shown in the FIGS., and is an inclusive term used to also apply to disposable boxes used initially to transport the PPE packets 10 configured to allow direct dispensing of the PPE packets 10 from the boxes. These disposable storage/dispenser boxes can serve as standalone tabletop dispensers, or be further positioned inside more permanent, frame-like wall mounted dispensers, and thus the dispenser 20 shown in the FIGS. is a representative dispenser not meant to limit the dispenser to just what is shown.

In an optional step (not shown in FIG. 1) the number of groups of PPE packets are counted and a same number of dispensers as groups of packets are preinstalled on a wall so as to be ready to receive the PPE packets. For instance, when the healthcare facility orders PPE packets, if the healthcare facility's purchaser notes that only small and medium sized gloves are used, the healthcare facility can preinstall two dispensers and load one with small sized PPE packets and the second one with medium sized PPE packets according to the method 50. Note too that the groups of packets can include different packet compositions; for instance, in a solo family medicine practice with one doctor and one physician's assistant, one dispenser may store and dispense packets with small sized gloves and masks, and another separate dispenser may store and dispense packets with small sized gloves, masks, gowns, hats, and booties within a same examination room.

In a last step, the healthcare professional requiring PPE reviews the descriptions of the PPE packet contents listed on the dispensers 20 and self-selects an appropriate PPE packet 10 from the appropriate dispenser by grasping the PPE packet from a dispensing opening 38b formed into the dispenser 50f.

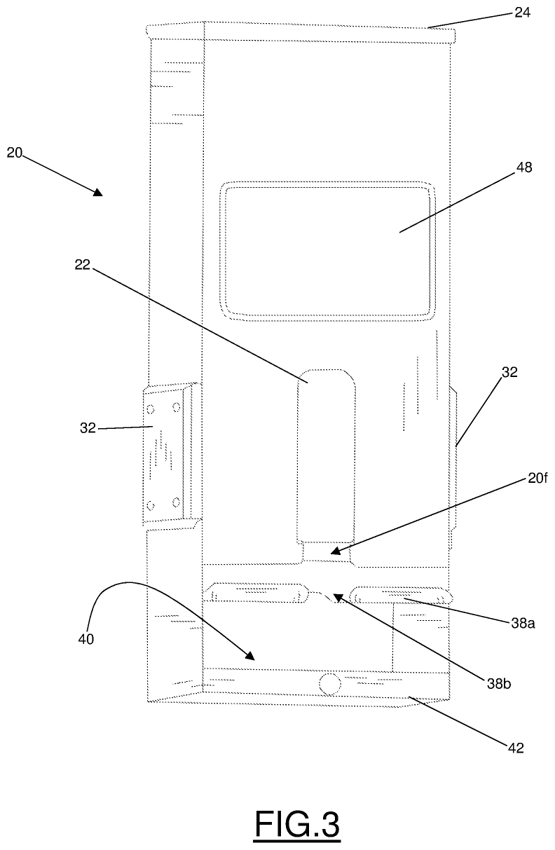

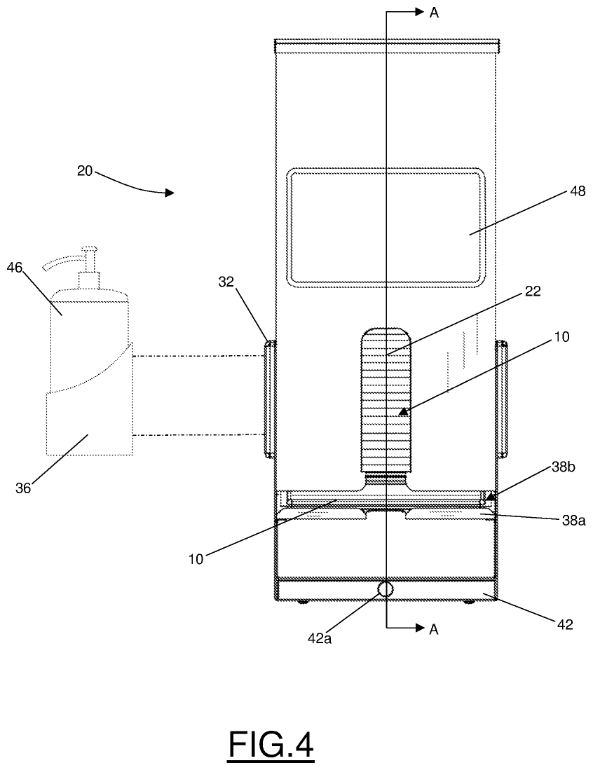

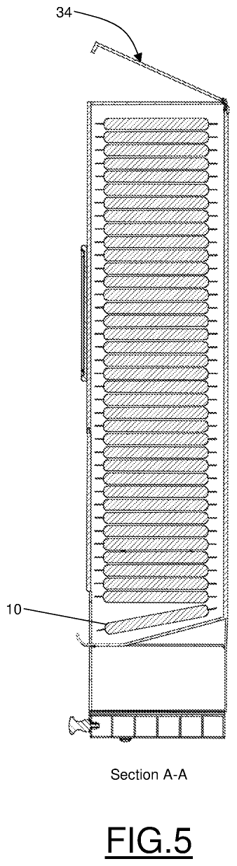

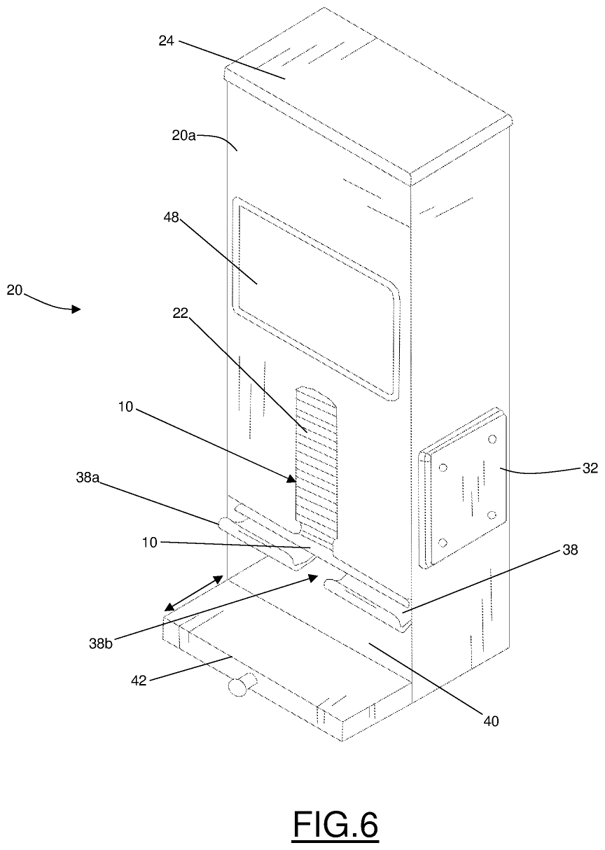

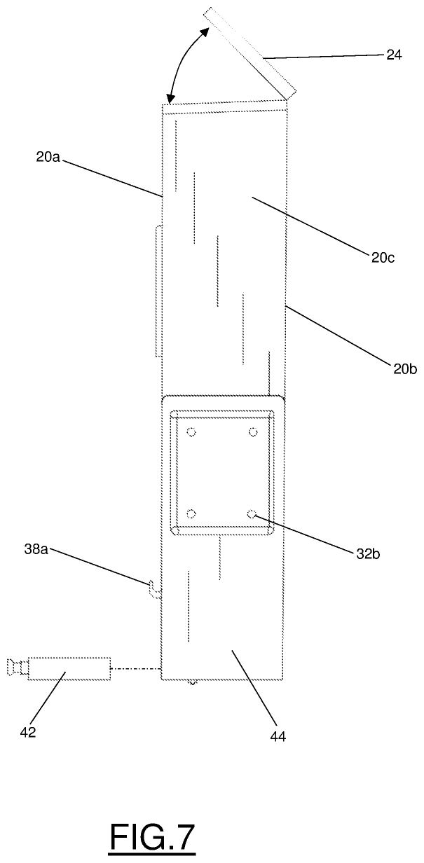

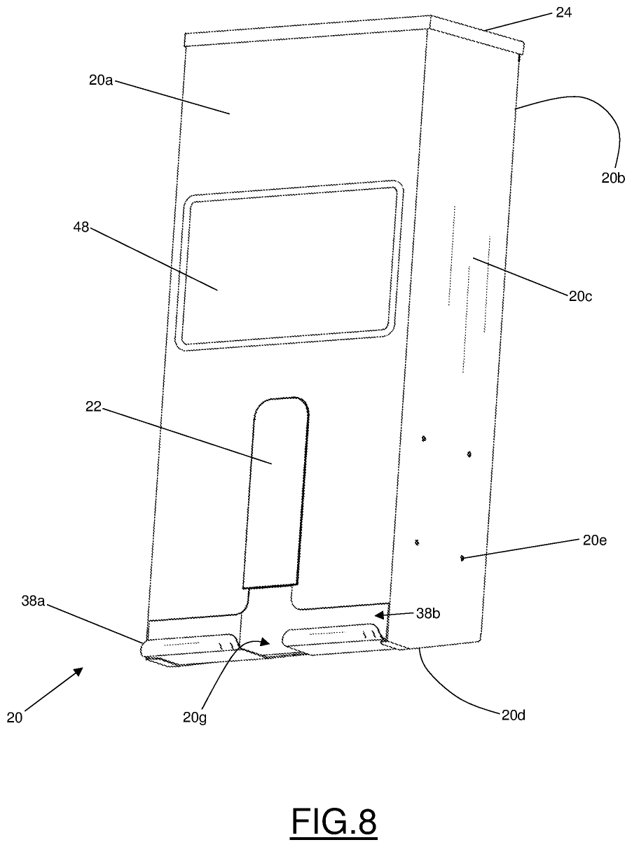

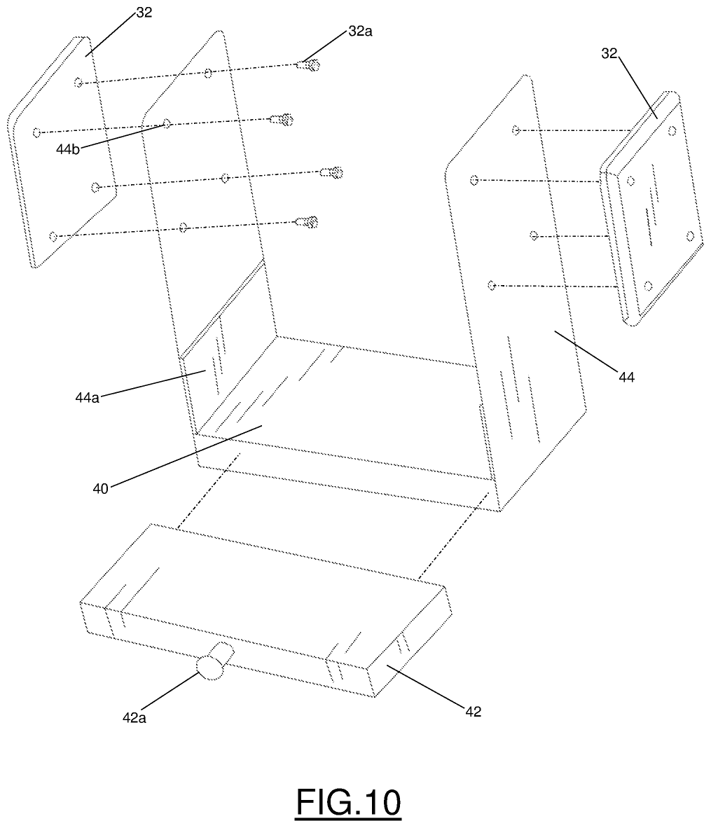

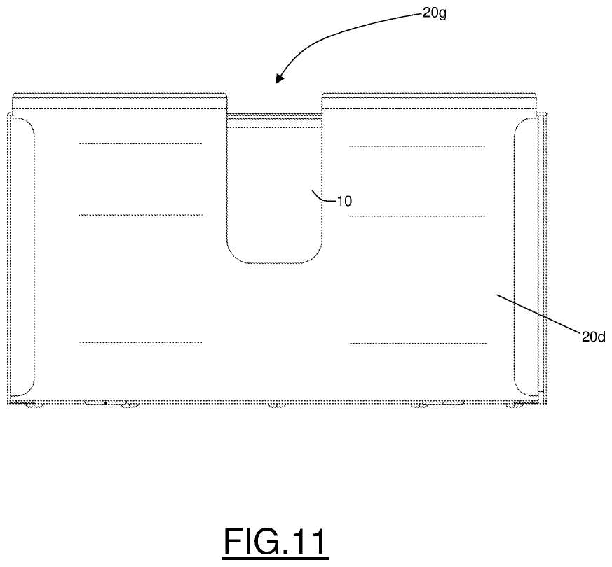

Turning to FIGS. 3-10, the dispenser 20 shown has a base dispenser unit 20 shown in FIGS. 8, 9 and 11, and an optional accessory shelf comprised of a U-shaped shelf frame 44, with a U-shaped frame insert 44a forming a lower shelf 40 and supporting a pull out shelf 42 with a knob 42a. The shelf frame 44 is further formed with apertures 44b that correspond in position and size with apertures 32b formed into an accessory plate 32. FIG. 10 shows the shelf frame 44 with a pair of accessory plates 32; note that the shelf frame 44 can be used with one or no accessory plates 32. The accessory plate 32 is a universal accessory mounting plate allowing a variety of different accessories to be mounted to the dispenser 20. For instance, in FIG. 4, a hand sanitizer bottle 46 and holder 36 are shown in exploded view with the accessory plate 32, the dispenser 20 and the shelf frame 44. Note that the accessory plate 32 can be used without the shelf frame 44 and directly fastened to the dispenser 20. In FIG. 10, fasteners 32a are typically fastened into the apertures 44b of the shelf frame 44, into the corresponding apertures 32b of the accessory plate 32 and then finally into the accessory itself, such the hand sanitizer holder 36 in FIG. 4. Other methods of fastening accessories to the dispenser can include other types of known male-female couplers, including pressure fitted couplers, and thus the fastening system shown in the FIGS. is meant to illustrate one useful way of attaching accessories to the dispenser 20 and is not meant to limit accessory attachment to just what is shown in the FIGS. The apertures 44b 32b and accessory plate 32 are designed to accommodate a number of different accessories, including additional shelves, and a variety of different holders for items such as pens, tongue depressors, etc. The accessory attached to the accessory plate could even be one or more hooks adapted to hold a purse or articles of clothing.

The pull out shelf 42 and lower shelf 40 are particularly useful because the inventor, who is a healthcare professional himself, has noted that when putting on PPE, there is often no place to temporarily store items such as cellphones and even patient files and thus there is an awkward juggling of items or searching for temporary storage surfaces while putting on PPE. Given that the PPE packets 10 contain several items, a place to temporarily store an opened PPE packet while putting on some of the components is useful and convenient.

Turning now to the dispenser 20, the representative dispenser shown in FIGS. 3-9 and 11 has a body with a rectangular cross section, with a front wall 20a, opposed back wall 20b and a pair of side walls 20c that have sloped lowermost edges, resulting in a floor 20d attached to the walls 20a 20b 20c at the lowermost end of the body. The floor 20d thus has a sloped profile, as shown most clearly in FIG. 8. This sloped floor 20d encourages gravity positioning of a lowermost PPE packet 10 in the dispenser 20 so as to be in a dispensing position at the dispenser opening 38b formed between a lowermost edge of the front wall 20a and an upper shelf 38, the upper shelf 38 being an upper side of the floor 20d facing inside an interior storage area 34 of the dispenser 20. The front wall 20a at its lowermost edge is further comprised of a front wall notch 20f formed below a transparent or semi-transparent lens 22. The floor 20d similarly is formed with a floor notch 20g aligned with the front wall notch 20f such that when the PPE packet 10 is dispensed, the notches 20f 20g allows for easier dispensing of the PPE packet 10 in the dispensing position at the dispenser opening 38. The floor 20d is further formed with a pair of upwardly curved lips 38a on either side of the floor notch 20g to maintain the PPE packet 10 in the dispensing position. The lips 38a could be a curved lip as shown in the representative embodiment, or some other type of physical stop with a size and shape sufficient to prevent the PPE packet 10 in the dispensing position from falling out of the dispenser opening 38b.

The dispenser 20 ideally includes means allowing the dispenser's PPE packet supply to be monitored. In the representative embodiment in the FIGS, the lens 22 allows the healthcare facility to easily monitor supplies in each dispenser 20 at a glance, but the inventor notes however that there are other ways of monitoring supplies, and the dispenser could also be configured with weight, volume or other sensors on the floor 20d or inside the interior storage area 34 that transmit information to the facility to alert the relevant people that supplies are running low, or to automatically order more supplies when a preset weight or other measuring parameter is met. Given that healthcare facilities are labor intensive, complicated businesses to run, such seemingly small changes, such as the method 50 and dispenser 20 described herein, can result in significant time and cost savings while improving care for patients. Even the elimination of a single step, such as monitoring the dispensers for PPE packet supply, can be a significant time and cost saving for the healthcare facility and free up healthcare professionals to do other things. While all facilities can benefit from this optional step and inclusion into the dispenser, smaller facilities will gain the most benefit, due to their sensitivity to labor costs and the need for even more operational efficiency.

The dispenser 20 is also configured to hold a content indicator or white board 48 or other indicator of dispenser contents. In the representative embodiment shown in the FIGS., the white board 48 is magnetic and can be removed from the dispenser 20 or reoriented as desired. The white board 48 could also be permanently affixed to the dispenser 20 or simply be an area on the front wall 20a suitable for stickers or other ways to label the contents of the dispenser 20.

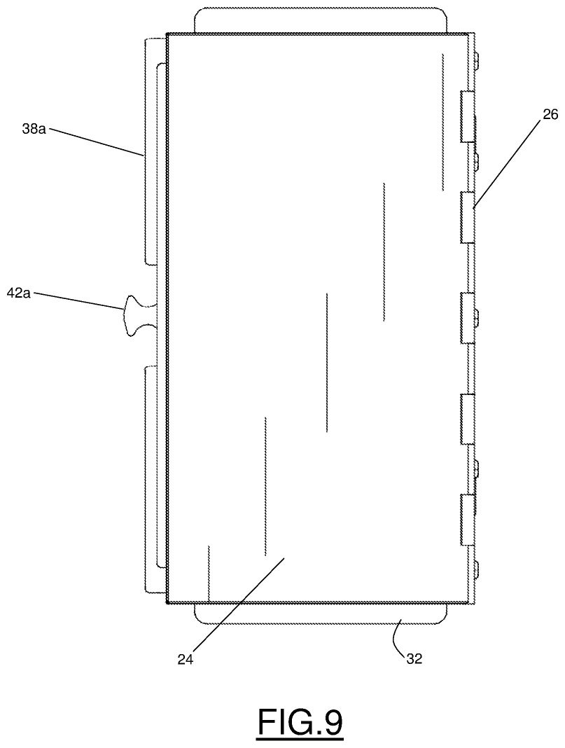

The dispenser 20 has a hinged lid 24, as shown best in FIG. 9, allowing for an interior storage area 34 to be easily supplied with PPE. In some cases, the healthcare facility may also wish to lock the dispenser 20 to avoid mischief by patients or others who may put inappropriate things into the dispenser 20, such as mixing up PPE packet sizes, and this can be easily achieved by modifying the lid 24 with known methods and apparatuses, such as installing lock brackets on the lid 24 and front wall 20a, for instance.

It is to be understood that the above-described arrangements are only illustrative of the application of the principles of the present invention. Numerous modifications and alternative arrangements may be devised by those skilled in the art without departing from the scope of the present invention.

While in the representative embodiments shown are for disposable PPE, the inventor notes that reusable PPE is also appropriately bundled and dispensed using the method 50 and dispenser 20 according to the invention, particularly in situations where there is limited storage space or when healthcare professionals need “grab and go” packets, which increases convenience. Given that discarded PPE is also a concern, packaging clean PPE and/or dispensing the same using the method 50 can also provide value to the healthcare facilities. For instance, reusable PPE for smaller surgical centers might benefit from bundling PPE packets, such as for in-patient care. For instance, when patients receive a drawstring bag for personal items, a reusable gown and knitted socks or foot coverings could easily be packaged and dispensed using the method 50, saving nurses from having to gather and distribute all these different items and thus increasing operational efficiency and uniformity in patient care. Healthcare facilities are always looking to improve their services, and the method 50 and dispenser 20 described herein can benefit these facilities by standardizing routine but time intensive activities. Hence, the inventor wishes to stress that the term PPE used in this disclosure covers both disposable and reusable personal protective equipment.

The inventor notes too that the terms gloves, masks, gowns, head coverings or caps, and shoe coverings or booties are general terms and refer to many different PPE protection level ratings. For instance, the term gown used in this disclosure refers to Rating Levels 1-4 gowns, whose level rating is based on a level of fluid and/or microbe barrier protection. A Level 1 gown has minimal fluid barrier protection and is used for daily patient care where there is little risk of fluid exposure. Level 2 has minimal to low fluid barrier protection and is used in situations where there is only a slight risk of fluid exposure, for instance during minimally invasive surgical procedures. Level 3 has moderate fluid barrier protection and is used for the widest range of surgical procedures where the risk of fluid exposure is moderate. Level 4 has the highest fluid and microbial barrier protection to protect against bloodborne pathogens in critical zones, and is used for long, fluid-intensive procedures. These levels are regulated and required by various government agencies. Hence, the type of PPE components in the selecting step of the method 50 disclosed herein also includes selecting PPE based on Levels 1-4 or other ratings as defined by the relevant regulatory agencies. Similar level ratings exist for head coverings, shoe coverings, masks, and gloves. In the United States, PPE may be subject to regulation by federal, state, and local governments.

Further, while the step of vacuum sealing creates slightly more packaging waste, the nature of vacuum sealing is such that the packaging is limited and will have a relatively small impact on the environment. The additional cost of bundling and dispensing is easily offset by reducing storage space and shipping costs, as well as reducing time required to supply and dispense PPE. Older facilities in particular will benefit from the method 50 and dispenser 20 according to the invention, because they already have limited space and thus can better use existing space without requiring expensive renovations. New facilities can better and more efficiently allocate space, using fewer construction materials, with smaller, more efficiently used spaces translating into lower operational costs. It is much more economical to heat, cool and power a smaller facility and thus more of the facility can be devoted to patient care rather than storage of PPE.

The inventor believes that a wall mounted dispenser is most useful in the healthcare facility setting given the limited space in most examination rooms, however door mounted dispensers or table top dispensers may also be suitable for use with the PPE packets made and dispensed by the method 50. The dispenser 20 can also be comprised of two or more dispensers 20 joined together, using the apertures 20e for instance to directly join the dispensers together, or a single dispenser body with multiple interior space areas 34 for housing different groups of PPE packets within a same dispenser, but keeping the groups separated and allowing for separate dispensing. The inventor notes that healthcare facilities are highly regulated and he believes that his dispenser 20 and method 50 can be adapted to comply with regulations worldwide, allowing all facilities in a regulated area to have a consistent and uniform PPE dispensing system, further simplifying operations and compliance.

I claim:

1. A method of bundling and dispensing personal protective equipment for dispensing by a dispenser, the method comprising the steps of:
selecting personal protective equipment components based on size and type;
bundling the selected personal protective equipment components according to the selected personal protective equipment component size and type; and
vacuum sealing the bundled personal protective equipment components to form individual personal protective equipment packets with a predetermined uniform size and shape;
wherein the size of the personal protective equipment components include small, medium, large, and single sizes;
wherein the type of personal protective equipment components includes masks, gloves, head coverings, gowns, and shoe coverings; and
wherein the type of personal protective equipment components includes disposable, reusable, or both disposable and reusable components.

2. The method in claim 1 further comprising the steps of:
grouping the personal protective equipment packets according to packet content;
loading each group of personal protective equipment packets into its own dispenser; and
dispensing personal protective equipment packets by grasping a personal protective equipment packet from its dispenser.

3. The method in claim 2, wherein the step of grouping is further comprised of sorting the personal protective equipment packets based on component size and component type.

4. The method in claim 1, wherein the personal protective equipment packets are planar with a flattened profile.

5. The method in claim 1, wherein the personal protective equipment packets have either a tear off strip or a notched edge formed into the packet, whereby the packets are opened by removing the tear off strip or tearing the packet along the notched edge.

6. The method in claim 1, wherein the step of selecting further comprises selecting at least a pair of gloves.

7. The method in claim 6, wherein the step of selecting further comprises selecting a mask.

8. The method in claim 6, wherein the step of selecting further comprises selecting a gown.

9. The method in claim 1, wherein the step of selecting is further comprised of selecting disposable components.

10. The method in claim 1, wherein the step of selecting is further comprised of selecting reusable components.

11. The method in claim 1, wherein the type of personal protective equipment components includes selecting components based on fluid or microbial barrier rating levels.

12. The method in claim 1, wherein the step of bundling is further comprised of stacking the personal protective equipment components in a same order prior to the step of vacuum sealing.

13. A personal protective equipment packet made by the method in claim 1, comprising personal protective equipment components having a small, medium, large, or single sized component and wherein the personal protective equipment packet includes types of personal protective equipment components that are disposable, reusable, or both disposable and reusable.

14. A dispenser for use with the method in claim 2, comprising:
a dispenser body having a front wall, a back wall, and a side wall defining an interior storage area sized and shaped to receive the personal protective equipment packets;
a dispensing opening formed into the dispenser body allowing access to the interior storage area.

15. The dispenser in claim 13, wherein the dispenser body is further comprised of a floor at its lowermost end and a lid at its uppermost end, and wherein the dispenser opening is a slot formed into the lowermost end of the dispenser body above the floor and configured as a through-opening allowing access to the interior storage area.

16. The dispenser in claim 13, wherein the front wall is at right angles with the side wall.

17. The dispenser in claim 13, wherein the floor is further comprised of a floor notch and a lip adjacent the dispenser slot.

18. The dispenser in claim 13, further comprising an accessory plate having at least one aperture and adapted to be affixed to both the dispenser body and to an accessory attached to the dispenser body.
